(19) United States
(12) Patent Application Publication
(10) Pub. No.: US 2015/0343162 A1
(43) Pub. Date: Dec. 3, 2015

(54) Mask with Tang Protrusions for Improved Grasping Ability

(71) Applicant: Medline Industries, Inc., Mundelein, IL (US)

(72) Inventors: Christian Svoboda, Chicago, IL (US); Derek Roberts, Chicago, IL (US)

(73) Assignee: Medline Industries, Inc., Mundelein, IL (US)

(21) Appl. No.: 14/294,806

(22) Filed: Jun. 3, 2014

(51) Int. Cl. A61M 16/06

(52) U.S. Cl. CPC A61M 16/0622; A61M 16/0683; A61M 2205/02; A61M 2205/586

(57) ABSTRACT

A mask (100) includes a mask body (101). The mask body (101) defines an inner surface (602) and an outer surface (102). The inner surface can define a concave chamber (801) suitable for placement over a patient's nose, mouth, or combination thereof. The outer surface (102) can define a first tang (104) protruding from the outer surface of the mask body, where the first tang defines a first concave finger receiving surface (304). Similarly, the second tang (105) can protrude from the outer surface of the mask body, where the second tang defines a second concave finger receiving surface (305).

### BACKGROUND

1. Technical Field

This disclosure relates generally to masks, and more particularly to masks for covering the nose and mouth.

2. Background Art

Facial masks are frequently used in medical procedures. For example, masks covering the nose and mouth can be used as respirators through which anesthesia or other medications can be delivered to a patient during medical procedures. Where used in this fashion, the mask is placed over the nose or mouth of a patient while anesthesia is delivered through a gas port. The patient inhales the anesthesia while an administration pump ensures the proper dosage is delivered.

A problem with prior art masks involves the seal between the patient's face and the mask. Where this seal is compromised, less than the proper dosage of anesthesia may be delivered to the patient. This can result in more time being required to anesthetize the patient, which delays medical treatment and increases costs. It would be advantageous to have an improved mask that was easier to seal against the face of a patient.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present disclosure.

FIG. 1 illustrates a perspective view of one explanatory mask in accordance with one or more embodiments of the disclosure.

FIG. 2 illustrates another perspective view of one explanatory mask in accordance with one or more embodiments of the disclosure.

FIG. 3 illustrates a side elevation view of one explanatory mask in accordance with one or more embodiments of the disclosure.

FIG. 4 illustrates a front elevation view of one explanatory mask in accordance with one or more embodiments of the disclosure.

FIG. 5 illustrates a rear elevation view of one explanatory mask in accordance with one or more embodiments of the disclosure.

FIG. 6 illustrates a bottom plan view of one explanatory mask in accordance with one or more embodiments of the disclosure.

FIG. 7 illustrates another side elevation view of another explanatory mask in accordance with one or more embodiments of the disclosure.

FIG. 8 illustrates a sectional view of one explanatory mask in accordance with one or more embodiments of the disclosure.

FIG. 9 illustrates another side elevation view of another explanatory mask in accordance with one or more embodiments of the disclosure.

FIG. 10 illustrates a top plan view of one explanatory mask in accordance with one or more embodiments of the disclosure.

FIG. 11 illustrates a user holding a prior art mask.

FIG. 12 illustrates a user holding one explanatory mask in accordance with one or more embodiments of the disclosure.

FIG. 13 illustrates another user holding one explanatory mask in accordance with one or more embodiments of the disclosure.

FIG. 14 illustrates a user applying one explanatory mask in accordance with one or more embodiments of the disclosure to the face of a patient.

FIG. 15 illustrates one explanatory method in accordance with one or more embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE DRAWINGS

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions. Also, reference designators shown herein in parenthesis indicate components shown in a figure other than the one in discussion. For example, talking about a device (10) while discussing figure A would refer to an element, 10, shown in figure other than figure A.

Embodiments of the present disclosure provide an improved mask that is easier to hold and easier to apply to a patient than are prior art designs. Moreover, in one or more embodiments a user can simply hold the mask with two fingers, and without squeezing or otherwise applying pressure to the mask. This two-finger handling is provided, in one embodiment, by a pair of tangs that extend distally from the mask body. The two tangs serve as “ledges” for the user's fingers, e.g., the user's thumb and forefinger. As the center of gravity of the mask is located beneath these ledges, gravity works to pull the mask body downward when the mask is held in an orientation in which it would be when being applied to a patient, thereby causing each tang to apply pressure to the top of the user's fingers. In effect, this advantageous use of gravity allows the mask to “hang” on the user's fingers without necessitating that the user apply lateral pressure to hold the mask. Advantageously, this frees up one, two, or three of the user's fingers for placement on a user's face to ensure that a high-integrity seal exists between the mask and the user's face.

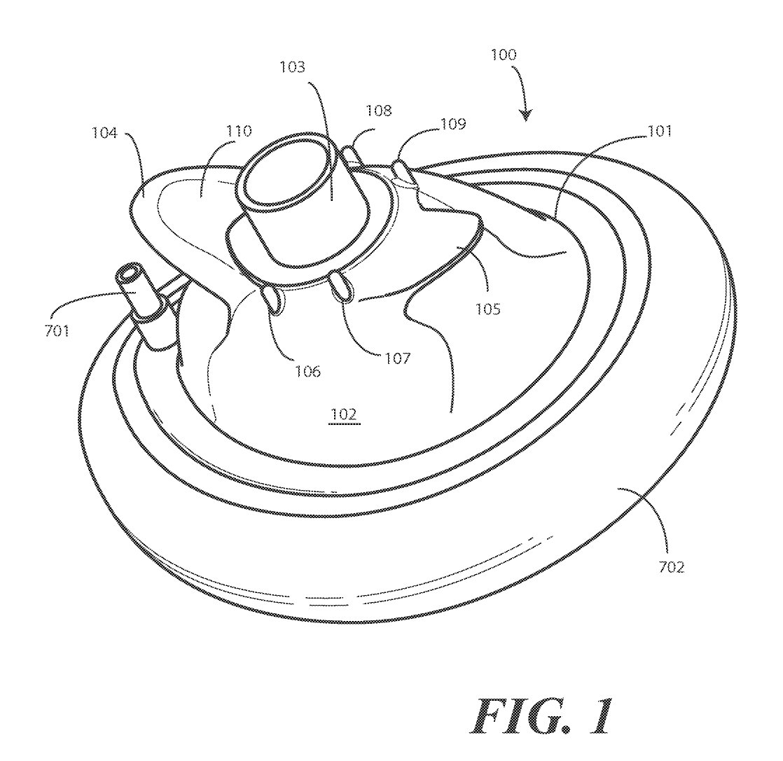

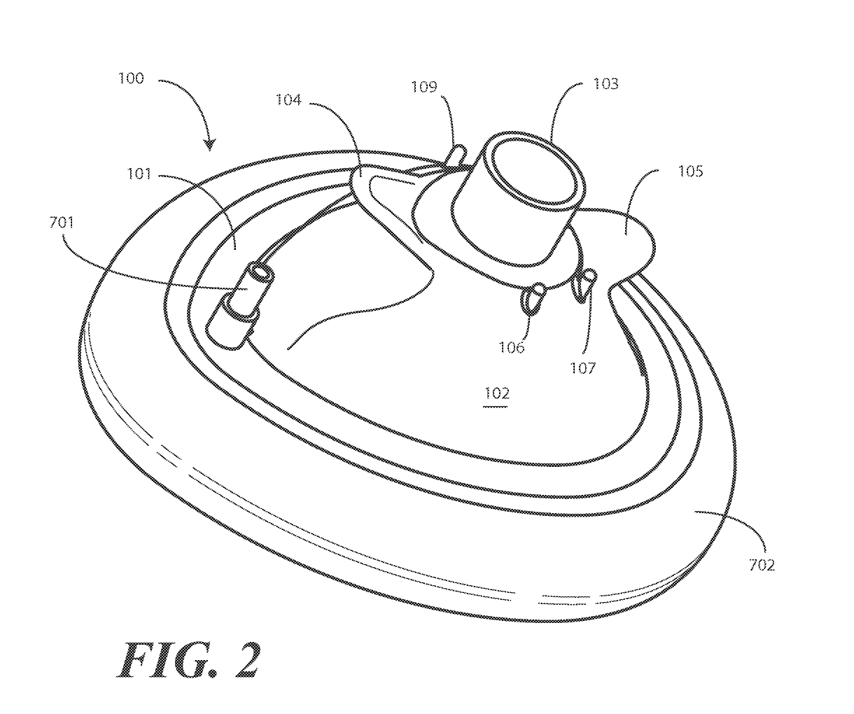

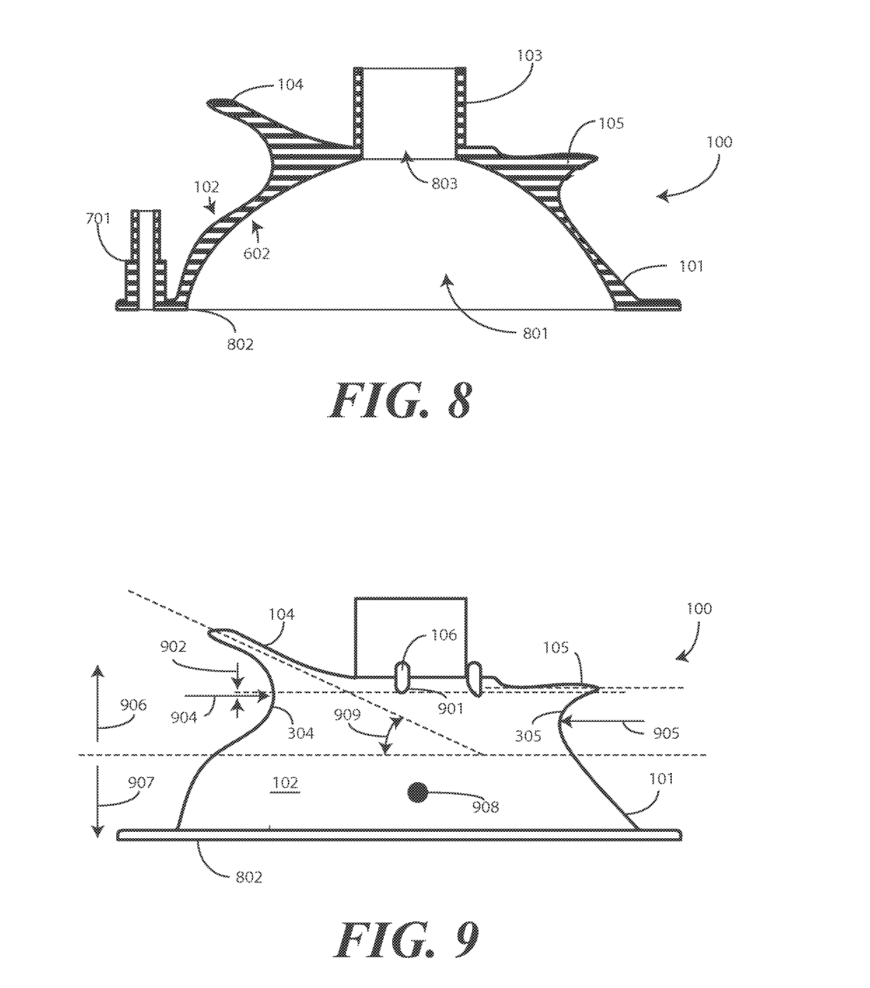

Turning now to FIGS. 1-8, illustrated therein is one embodiment of a mask 100 in accordance with one or more embodiments of the disclosure. The mask 100 includes a mask body 101, which has an outer surface 102 and an inner surface 602. As best shown in FIG. 8, in one embodiment the inner surface 602 defines a concave chamber 801 that terminates at a rim 802. A patient's nose and mouth can be positioned in the concave chamber 801 to receive oxygen, anesthesia, or other gasses through the nose and mouth.

In one embodiment, the mask body 101 is manufactured from a thermoplastic by way of an injection molding process. For example, in one embodiment the mask body 101 is manufactured from polycarbonate. Those of ordinary skill in the art having the benefit of this disclosure will understand that other thermoplastic materials, such as styrene, ABS, polycarbonate-ABS, and so forth, can be used for the mask body 101 as well.

Different materials can impart different mechanical characteristics to the mask body 101. For example, in one embodiment, the mask body 101 is manufactured from a material that causes the mask body 101 to be flexible. In another embodiment, the mask body 101 is manufactured from a material that causes the mask body 101 to be semi-rigid. In yet another embodiment, the mask body 101 is manufactured from a material that causes the mask body 101 to be rigid. Different materials can be used as well to make the outer surface 102 have varying degrees of friction as well. For example, one material may make the outer surface 102 slick and glossy, while another material may make the outer surface 102 have a high coefficient of friction so as to more readily stay within a user's hand when held.

In one embodiment, the mask body 101 is formed as a unitary, contiguous part. Said differently, in one embodiment the various features that will be described below are all integrally formed into a single part that defines by the mask body 101. In other embodiments, some or all of the features of the mask body 101 can be separable components. Illustrating by example, as will be described in more detail with reference to FIG. 7 below, in one embodiment a lumen 701 that can optionally be included for inflation of an inflatable bladder 702 can be selectively detachable from the mask body 101.

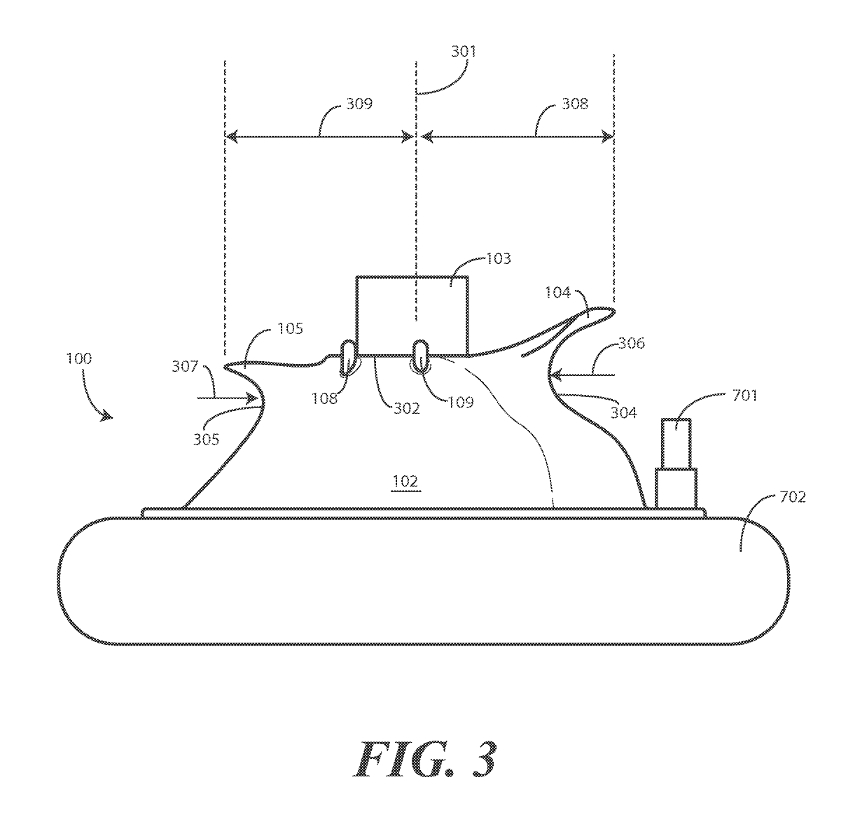

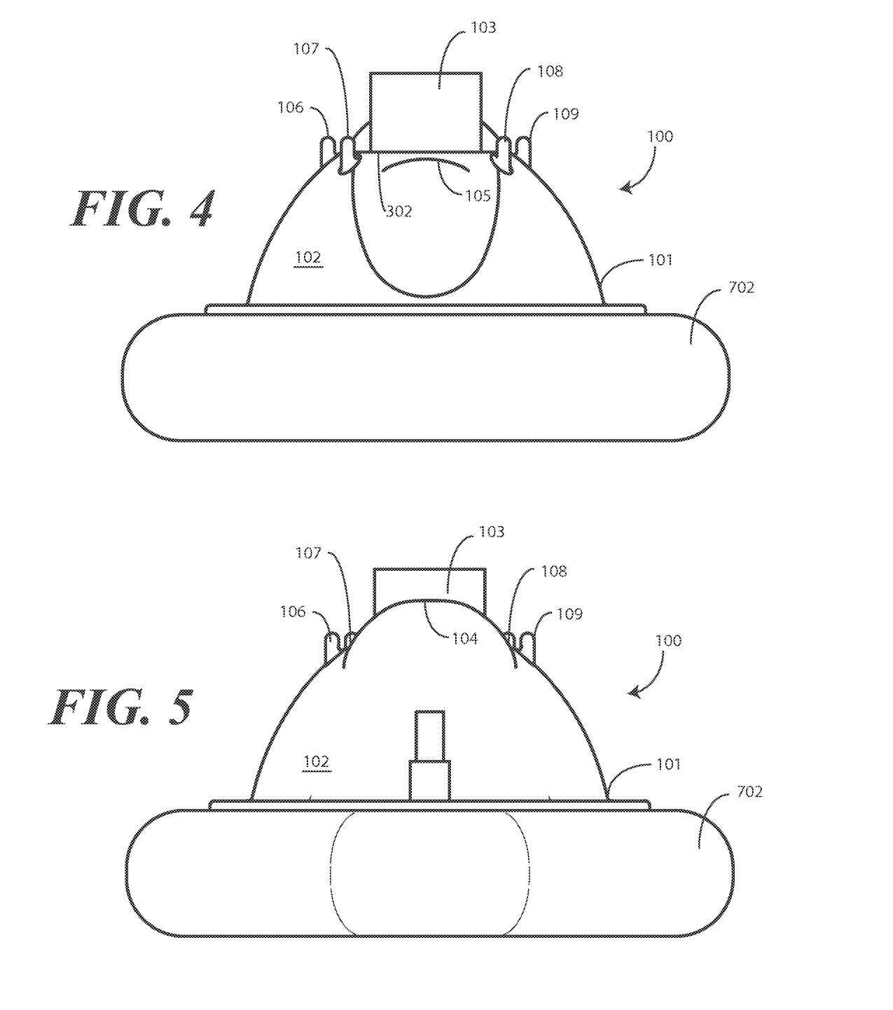

In one or more embodiments, the mask body 101 includes a gas port 103. In one embodiment, the gas port 103 is cylindrical, centered about an axis 301. It should be noted that the gas port 103 can take other shapes, as necessary to couple to a gas hose. Further, it should be noted that the gas port 103 can include threads, snaps, or other form factor features for coupling to the gas hose. As best shown in FIGS. 3-4, in one embodiment the gas port 103 extends distally from an apex 302 of the mask body 101 upward and away from the mask body 101.

In one embodiment, the gas port 103 is configured as a hollow tube that terminates at an aperture 803 that forms an entryway into the concave chamber 801. In one or more embodiments, not only does the gas port 103 deliver oxygen, anesthesia, or other gasses to a patient, but it also removes exhaled air by way of a vacuum pump operable with the hose coupled to the gas port 103. This process is sometimes referred to as “scavenging” exhaled air through the same gas port 103 with which gasses are delivered to the patient.

In one or more embodiment, the outer surface 102 of the mask body 101 defines features that make the mask 100 easier to hold. In the illustrative embodiment of FIGS. 1-8, these features include a first tang 104 and a second tang 105. The first tang 104 protrudes from the outer surface 102 of the mask body 101. Similarly, the second tang 105 protrudes from the outer surface 102 of the mask body 101.

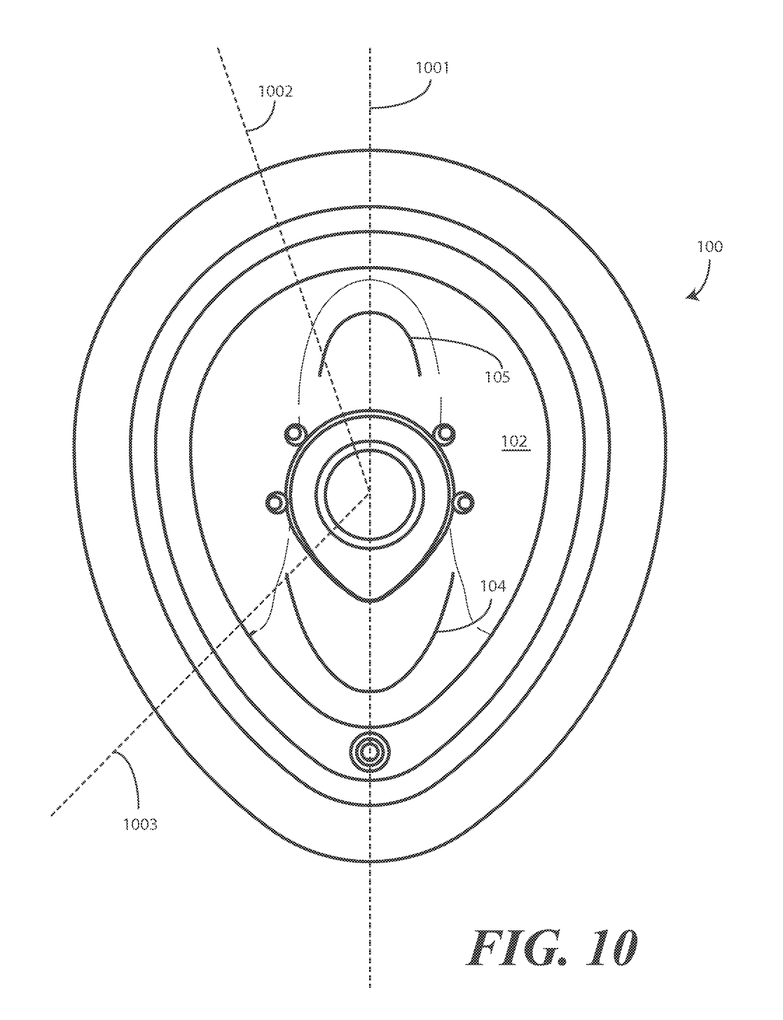

In one embodiment, the first tang 104 and the second tang 105 extend distally from the mask body 101 in opposite directions. Turning briefly to FIG. 10, in one embodiment the first tang 104 and the second tang 105 extend from the outer surface 102 of the mask body 101 in opposite directions along an extension axis 1001. In FIG. 10, the first tang 104 extends downward on the page, i.e., in the negative direction along the extension axis 1001, while the second tang 105 extends upward on the page, i.e., in the positive direction along the extension axis 1001. This results in the first tang 104 and the second tang 105 being disposed substantially 180 degrees about the mask body 101 relative to each other in this embodiment. In other embodiments, this angle can be greater than, or less than, 180 degrees. For example, the embodiment of FIG. 10 is well suited for ambidextrous use as a user can hold the mask 100 with either the right hand or the left hand due to the 180-degree alignment. However, in a more customized design, such as one specifically made for left-handed people, one or both of the first tang 104 or the second tang 105 may be disposed to the left of the extension axis 1001 shown in FIG. 10, which results in two extension axes 1002,1003 having an angle of less than 180 degrees when measured interior to the extension axes 1002,1003. The opposite, of course, could be true with one or both of the first tang 104 or the second tang 105 being disposed to the right of the extension axis 1001 shown in FIG. 10.

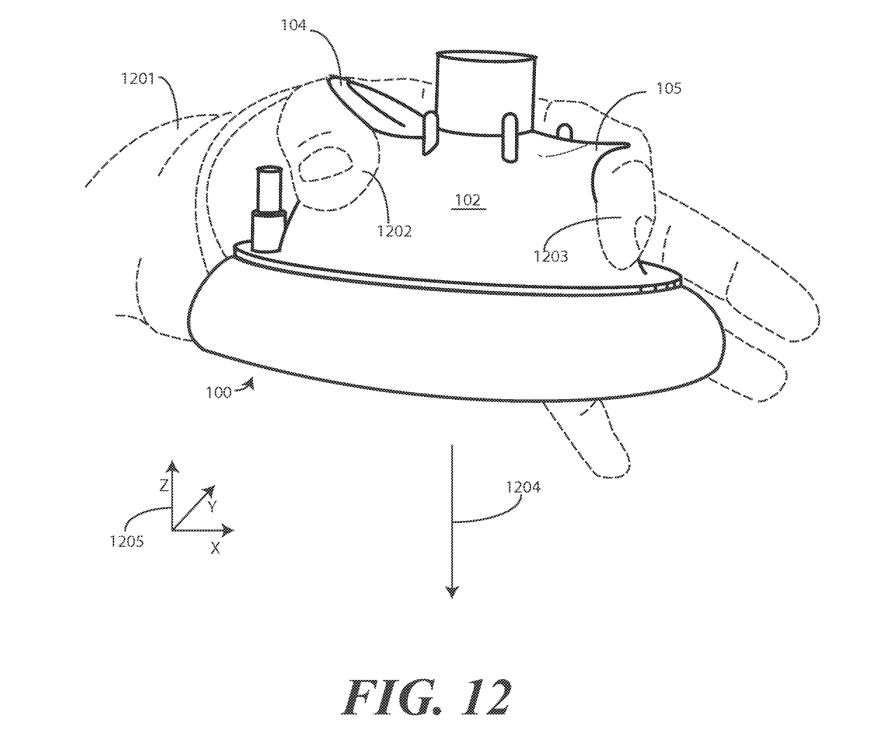

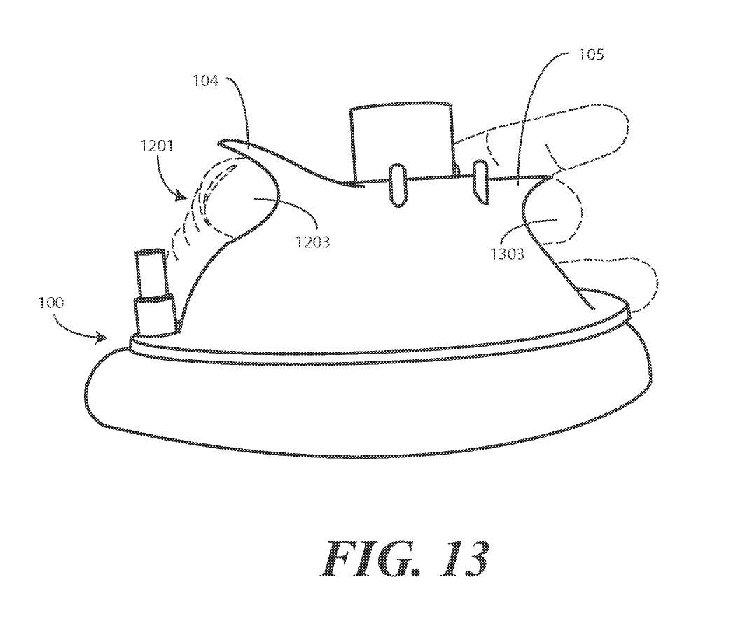

Turning now back to FIGS. 1-8, in one embodiment each tang defines a finger receiving surface that is concave relative to the outer surface 102 of the mask body 101. As best viewed in FIG. 3, in one embodiment the first tang 104 defines a first concave finger receiving surface 304. Similarly, the second tang 105 defines a second concave finger receiving surface 305. In one or more embodiments, each concave surface can be defined by a radius corresponding to an average user's finger size. For example, in one embodiment the first concave finger receiving surface 304 is defined by a first radius 306 that is complementary to one or more of a user thumb, a user palm, or a user web disposed between the user thumb and the user palm. Illustrating by example, in one embodiment the first concave finger receiving surface 304 is defined by a first radius 306 that is complementary to a user thumb. This allows the user thumb to conveniently seat within the first concave finger receiving surface 304 with the first tang 104 disposed atop the user thumb. Similarly, in one embodiment the second concave finger receiving surface 305 can be defined by a second radius 307 that, in one embodiment, is complementary to a user forefinger. This allows the user forefinger to conveniently seat within the second concave finger receiving surface 305 with the second tang 105 disposed atop the user forefinger. When the mask 100 is lifted—upward along the page as viewed in FIG. 3, gravity pulls the mask 100 downward, such that gravity acts to press the first tang 104 against the user thumb and the second tang 105 against the user forefinger. This feature, which will be shown in more detail with reference to FIGS. 12-13 below, allows the user to conveniently to support the mask 100 on the user thumb and the user forefinger without user thumb pressure or user forefinger pressure applied to the outer surface 102.

In one or more embodiments, the gas port 103 is disposed between the first tang 104 and the second tang 105. In one or more embodiments, the first tang 104 and the second tang 105 extend distally from the outer surface 102 different distances. As best viewed in FIG. 3, in one embodiment the first tang 104 extends distally from the axis 301 of the gas port 103 a first distance 308, while the second tang 105 extends distally from the axis 301 of the gas port 103 a second distance 309. This results in the second tang 105 protruding distally from the outer surface 102 of the mask body 101 less than the first tang 104 in this illustrative embodiment.

This different distance protrusion serves multiple functions. First, by having the first tang 104 extend distally from the outer surface 102 of the mask body 101 more than the second tang 105, the first tang 104 can serve as a lifting ledge that seats atop a user thumb or web between a user palm and a user thumb when the mask is lifted. This additional support on larger portions of the user hand makes for easy lifting. Second, the different distance protrusion serves as mnemonic indicator of an instruction regarding how the mask 100 should be held. The mnemonic indicator causes the user to subconsciously grab the mask 100 with the larger tang disposed toward larger portions of the user hand, and the shorter tang disposed away from the user about the smaller user finger. While this illustrative embodiment employs tangs extending distally from the outer surface 102 of the mask body 101 by different amounts, those of ordinary skill in the art having the benefit of this disclosure will appreciate that the tangs could extend from the outer surface 102 of the mask body 101 by the same amount as well.

As briefly mentioned above, in one or more embodiments, the mask body 101, which is shown separately in FIG. 8 (and also in FIG. 9 below), is selectively attachable at the rim 802 to an inflatable bladder 702. A lumen 701 can be connected to the inflatable bladder 702 and can be used to electively inflate the inflatable bladder 702. In one embodiment, the inflatable bladder 702 includes an inner surface that defines an air chamber. A gas inlet (not shown) can be provided to connect the air chamber to the lumen 701 for the selective inflation and deflation of the inflatable bladder 702. A hose can be connected to the lumen 701 to deliver air through the lumen 701 to inflate the inflatable bladder 702.

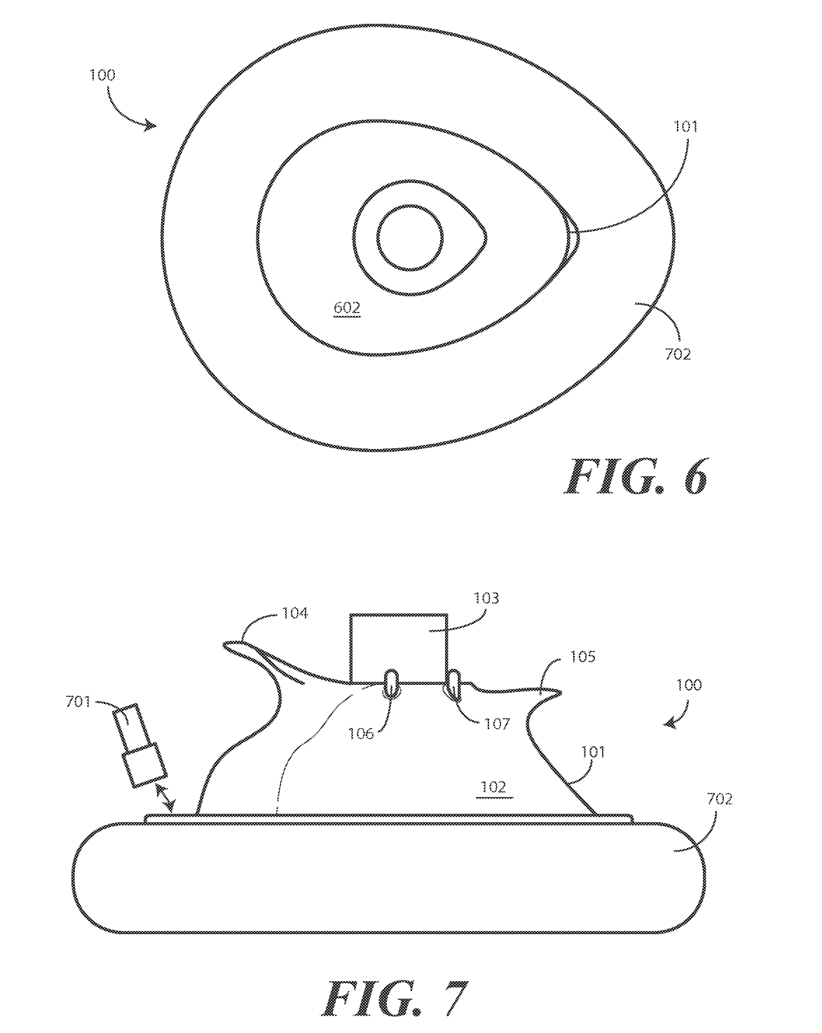

In one embodiment, the inflatable bladder 702 is generally toroidal in shape, thereby defining a toroidal inflatable bladder. It should be noted that in one or more embodiments, the toroidal inflatable bladder is not perfectly toroidal. As best shown in FIG. 6, in one embodiment the inflatable bladder 702 includes a mouth covering portion (the left side of the inflatable bladder 702 as viewed in FIG. 6) and a nose covering portion (the right side of the inflatable bladder 702 as viewed in FIG. 6). In one embodiment, the nose covering portion tapers, and thus has a corresponding tighter radius than does the mouth covering portion. Accordingly, as used herein, “toroidal inflatable bladder” is understood to include such shapes that provide advantageous coupling for a patient's face. In one or more embodiments, the lumen 701 is coupled to the inflatable bladder 702 along the mouth covering portion.

In one embodiment, the lumen 701 is integrally formed with the mask body 101. For example, in one embodiment the lumen 701 can be an extension of the mask body 101. In another embodiment, shown illustratively in FIG. 7, the lumen 701 can be selectively detachable from the mask body 101. In other embodiments, the lumen 701 may be a separate part from the mask body 101, but may be perdurably coupled thereto. For example, in one embodiment the lumen 701 is adhesively coupled to the mask body 101. In still other embodiments, such as where a patient face coupling device other than an inflatable bladder 702 is used, the mask body 101 may have no lumen at all. Such an embodiment is shown illustratively in FIG. 9.

In one or more embodiments, one or more posts 106,107,108,109 extend distally from the outer surface 102 of the mask body 101. The one or more posts 106,107,108,109, which are integrally formed with the mask body 101 in one or more embodiments, are useful to retain the mask 100 to a patients face. Specifically, one or more elastic ties can be looped around the one or more posts 106,107,108,109 to secure the mask 100 to the patient's head. In one embodiment the one or more posts 106,107,108,109 extend distally from the outer surface 102 of the mask body 101 substantially parallel to the axis 301 of the gas port 103.

In one embodiment, the plurality of posts 106,107,108,109 comprising at least two posts 106,107 disposed to a first side of the extension axis (1001) of the first tang 104 and the second tang 105. Similarly, at least two other posts 108,109 are disposed to a second side of the extension axis (1001). Experimental testing has shown this configuration works well in practice to use a contiguous loop of elastic material to secure the mask 100 to a patient's face. The contiguous loop (not shown) can be looped about two posts 106,107, wrapped about the patient's head, and then looped about the other two posts 108,109 to retain the mask 100 to the user's face.

The posts 106,107,108,109 and other features of the mask 100 are strategically located in one or more embodiments. Turning now to FIG. 9, illustrated therein are some advantageous locations for the various components of the mask 100.

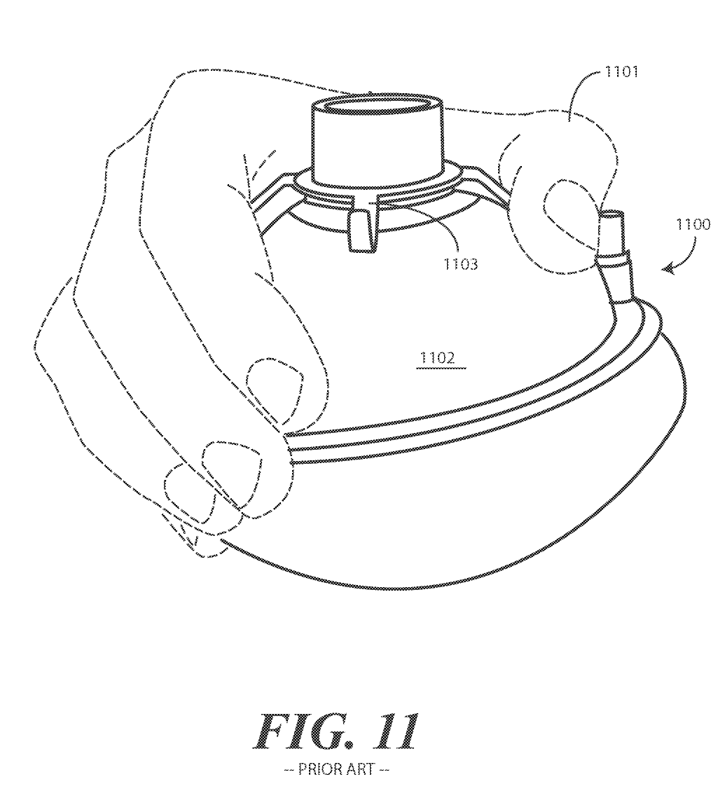

Continuing with the discussion of the posts 106,107,(108,109), embodiments of the disclosure contemplate that prior art masks using similar elastic material attaching devices suffer from problems. Turning briefly to FIG. 11, a user 1101 is shown holding a prior art mask, which was described in US Published Patent Application No. 2010/0199996 to Fu, which is incorporated herein by reference. The only way to satisfactorily hold this mask 1100 with one hand is to exert large amounts of lateral pressure against the outer surface 1102 of the mask 1100 to squeeze it. Testing has shown that the amount of pressure employed in the squeeze is so great that the mask 1100 actually deforms when squeezed sufficiently to retain the mask in the hand. Advantageously, as will be described in more detail with reference to FIGS. 12-13 below, the first tang (104) and second tang (105) of embodiments of the disclosure eliminate the necessity of this gorilla-type squeeze.

Another problem is shown in FIG. 11, however. Note that when the user 1101 squeezes the mask 1100, the hand must be placed atop posted disposed on a strap ring 1103. This is not only painful to the user 1101, but can result in the mask 1100 slipping from the user's hand. Where the mask 1100 must be sterile prior to application to a patient, an inadvertent drop can result in breach of the sterile field. Thus, the strap ring 1103 not only hurts when the user 1101 is holding the mask 1100, but it can result in a loss of sterility. Moreover, as the user's hand is atop one or more of the posts of the strap ring 1103, when the user 1101 is applying the mask 1100 to a patient, the user 1101 is unable to loop an elastic strap about the covered posts.

Turning now back to FIG. 9, embodiments of the present disclosure solve these problems. Recall from above that in one or more embodiments the first tang 104 and the second tang 105 serve as ledges that sit atop a user's thumb and forefinger. To avoid the covering problem shown in FIG. 11, in one embodiment, the posts 106,107,(108,109) are disposed above and/or about an anticipated hand location defined by the location of the first tang 104 and the second tang 105. In one embodiment at least one post, e.g., post 106 extends from a location 901 of the outer surface 102 superior 902 to an innermost surface 904,905 of either the first concave finger receiving surface 304 or the second concave finger receiving surface 305 relative to a medial plane 903 parallel to the rim 802 at which the concave chamber (801) terminates. This position advantageously places the posts 106,107,(108,109) above or about the anticipated hand location in one or more embodiments.

Other features are shown in FIG. 9 as well. For example, in this illustrative embodiment both the first tang 104 and the second tang 105 are disposed to a first side 906 of the medial plane 903. At the same time, the center of gravity 908 of the mask 100 is disposed to a second side 907 of the medial plane 903. This means that when a user picks up the mask 100 with a user thumb disposed along the first concave finger receiving surface 304 and a user forefinger disposed along the second concave finger receiving surface 305, the user can lift the mask 100 with the first tang 104 and the second tang 105 acting a ledges above the user thumb and user forefinger to advantageously allow gravity to act on the center of gravity to keep the mask 100 stable without user thumb pressure or user forefinger pressure applied to the outer surface 102 of the mask 100.

In one or more embodiments, one or both of the first tang 104 and the second tang 105 can extend from the outer surface 102 of the mask body 101 at different angles. For instance, at least one of the first tang 104 or the second tang 105 can protrude from the outer surface 102 at an acute angle 909 relative to the medial plane 903. In the illustrative embodiment of FIG. 9, the first tang 104 extends from the outer surface 102 at an acute angle 909 relative to the medial plane 903, while the second tang 105 protrudes from the outer surface substantially parallel to the medial plane 903. Testing has shown that this configuration not only feels comfortable in a user's hand, but also serves as the mnemonic indicator discussed above. Those of ordinary skill in the art having the benefit of this disclosure will understand that other protrusion angles for the first tang 104 and the second tang 105 can be used as well. For example, both the first tang 104 and the second tang 105 can extend at acute angles from the medial plane 903. Alternatively, the first tang 104 and second tang 105 can extend substantially parallel to the medial plane 903.

Turning now to FIGS. 11 and 12, illustrated therein is a user 1201 holding a mask 100 configured in accordance with one or more embodiments of the disclosure. As shown in FIG. 12, the user 1201 has placed a user thumb 1202 and a user forefinger 1023 along the first concave finger receiving surface (304) and the second concave finger receiving surface (305) respectively. The first tang 104 is disposed atop the user thumb 1202, while the second tang 105 is disposed atop the user forefinger 1203. As shown in FIG. 13, to provide an illustration of one alternate method of holding the mask 100, the user 1201 has placed a user thumb 1202 along the first concave finger receiving surface (304), while placing a user ring finger 11303 along the second concave finger receiving surface (305) respectively. This results in the first tang 104 being disposed atop the user thumb 1202, while the second tang 105 is disposed atop the user ring finger 1303. In either case, with the tangs functioning as ledges above the user fingers, the user 1201 is able to hold the mask 100 with no lateral pressure. For example, in FIG. 12, the first tang 104 and the second tang 105 support the mask 100 on the user thumb 1202 and the user forefinger 1203 without user thumb pressure or user forefinger pressure applied to the outer surface 102. The same is true in FIG. 13, but with the user ring finger 1303 substituted for the user forefinger 1203.

This ease of grasping is the result, in one embodiment, of the configuration of the first tang 104 and the second tang 105. The first tang 104 and the second tang 105 support the mask 100 on the user thumb 1202 and the user forefinger 1203 (or alternatively the user ring finger 1303) when the first tang 104 is disposed above the user thumb 1202 and the second tang 105 is disposed above the user forefinger 1203 (or alternatively the user ring finger 1303). This is true because gravity 1204 acts to press the first tang 104 against the user thumb 1202 and the second tang 105 against the user forefinger 1203 (or alternatively the user ring finger 1303). As best seen in FIG. 12, gravity 1204 acts in a negative direction along the Z-axis 1205, which is oriented radially from the center of the earth. When the user 1201 lifts the mask 100 in a positive direction along the Z-axis 1205, gravity 1204 pulls the mask 100 downward against the user's fingers to conveniently and effortlessly stabilize the mask in the user's hand. Note that no lateral force is required, which means that the gorilla squeeze required with the prior art mask (1100) of FIG. 11 is not required with masks configured in accordance with embodiments of the present disclosure.

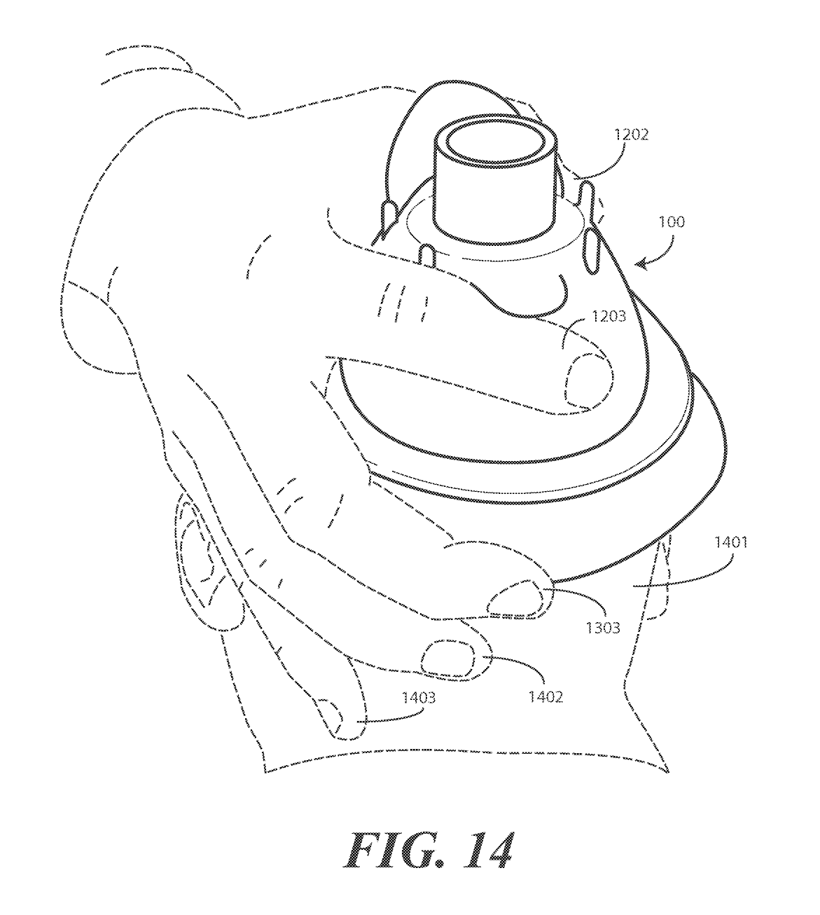

A second advantage is provided by embodiments of the disclosure. This second advantage is illustrated in FIG. 14. Turning now to FIG. 14, regardless of whether the mask 100 is held with the user thumb 1202 and user forefinger 1203, or alternatively the user ring finger (1303), only two fingers are required to securely hold the mask 100. This frees up one, two, or three fingers to perform other tasks. In the embodiment of FIG. 14, three of the user's fingers, i.e., user ring finger 1303, fourth finger 1402, and pinky finger 1403, are free and are not required to hold the mask 100. Accordingly, when the mask 100 is applied to a patient 1401, those free fingers can grasp the user's chin to ensure that a seal between the mask 100 and the patient 1401 has the necessary integrity to prevent leakage of oxygen, anesthesia, or other gasses being delivered to the patient. Further, this touching of the chin with the free fingers is comforting to the patient 1401 and prevents the mask from moving as the patient 1401 drifts into an anesthetized state during a medical procedure.

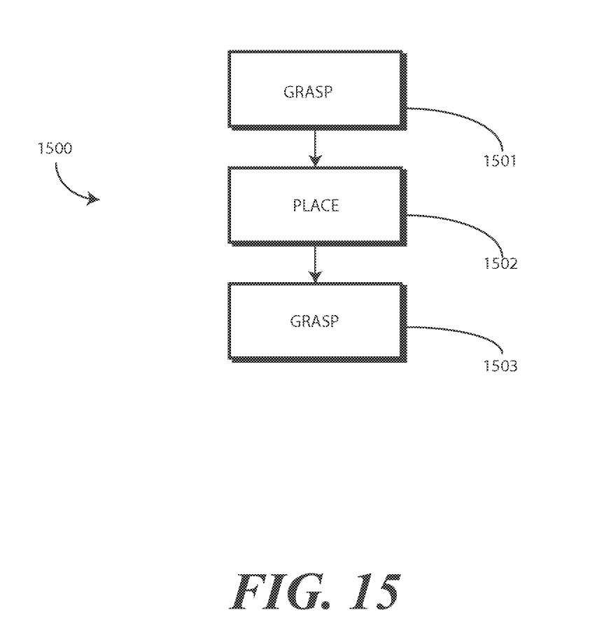

Turning now to FIG. 15, illustrated therein is one explanatory method 1500 of using a mask configured in accordance with one or more embodiments of the disclosure. Many of the steps of the method 1500 have been shown and described in the figures above, with special reference to FIGS. 12-14. However, FIG. 15 provides a more concise flow chart illustrating how an explanatory user may use a mask in accordance with embodiments of the disclosure.

At step 1501, the method 1500 includes grasping a mask body. In one embodiment, the mask body includes an inner surface and an outer surface. In one embodiment, the outer surface of the mask body defines a first tang protruding from the outer surface, wherein the first tang defines a first concave finger receiving surface. In one embodiment, the outer surface further defines a second tang protruding from the outer surface of the mask body and defining a second concave finger receiving surface. In one embodiment, the step 1501 of grasping comprises placing a thumb in the first concave finger receiving surface and a finger in the second concave finger receiving surface. The finger, as shown above, can be a forefinger, ring finger, or other finger.

At step 1502, the method 1500 places the mask body over one or more of a mouth or a nose of a patient. Since only two fingers were required to hold the mask at step 1501, one or more other fingers are free for other tasks. Thus, in one embodiment the method 1500 optional includes step 1503, in which the chin of a patient is grasped with one or more fingers other than the thumb and the finger.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited.

Numerous modifications, changes, variations, substitutions, and equivalents will occur to those skilled in the art without departing from the spirit and scope of the present disclosure as defined by the following claims. For example, while some features of masks are functional, these and other features can have an ornamental appearance as well.

Illustrating by example, in one embodiment, as best shown in FIG. 1, the first tang 104 is scalloped with the inclusion of a recess 110. This recess 110 can assist a user in pinching the first tang 104 to hold the mask 100 from one side. At the same time, this recess 110, as well as the first tang 104 itself, can impart ornamental aspects to the mask 100 as well. In one or more embodiments, the first tang 104 and the second tang 105 can be configured so that their ornamental appearance resembles the fins of a shark, thereby converting the mask 100 into an exciting and aesthetically pleasing device colloquially referred to as a “shark mask.” As the various features can provide unique ornamental appearances to the overall mask 100, design patents may be filed by the Applicant of the present application to claim the unique, novel, and non-obvious ornamental features.

Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims.

What is claimed is:

1. A mask, comprising:
a mask body comprising an inner surface and an outer surface; and
a gas port;
the inner surface defining a concave chamber;
the outer surface of the mask body defining:
a first tang protruding from the outer surface of the mask body, the first tang defining a first concave finger receiving surface; and
a second tang protruding from the outer surface of the mask body, the second tang defining a second concave finger receiving surface.

2. The mask of claim 1, the first tang and the second tang extending in opposite directions along an extension axis.

3. The mask of claim 2, the gas port extending distally from an apex of the mask body between the first tang and the second tang.

4. The mask of claim 1, the second tang protruding distally from the outer surface of the mask body less than the first tang.

5. The mask of claim 4, the first concave finger receiving surface defined by a first radius complementary to a user thumb.

6. The mask of claim 5, the second concave finger receiving surface defined by a second radius complementary to a user forefinger.

7. The mask of claim 6, the first tang and the second tang to support the mask on the user thumb and the user forefinger when the first tang is disposed above the user thumb and the second tang is disposed above the user forefinger such that gravity acts to press the first tang against the user thumb and the second tang against the user forefinger.

8. The mask of claim 7, the first tang and the second tang to support the mask on the user thumb and the user forefinger without user thumb pressure or user forefinger pressure applied to the outer surface.

9. The mask of claim 6, the concave chamber terminating at a rim, both the first tang and the second tang disposed to a first side of a medial plane parallel to the rim, a center of gravity of the mask disposed to a second side of the medial plane.

10. The mask of claim 9, the rim to attach to a toroidal inflatable bladder, the mask body further comprising a lumen to inflate the toroidal inflatable bladder.

11. The mask of claim 9, at least one of the first tang or the second tang to protrude from the outer surface at an acute angle relative to the medial plane.

12. The mask of claim 11, at least another of the first tang or the second tang to protrude from the outer surface substantially parallel relative to the medial plane.

13. The mask of claim 1, further comprising a plurality of posts extending distally from the outer surface.

14. The mask of claim 13, each post extending distally from the outer surface substantially parallel to an axis of the gas port.

15. The mask of claim 14, the plurality of posts comprising at least two posts disposed to a first side of an extension axis of the first tang and the second tang and at least two other posts disposed to a second side of the extension axis.

16. The mask of claim 13, at least one post extending from a location of the outer surface superior to an innermost surface of either the first concave finger receiving surface or the second concave finger receiving surface relative to a medial plane parallel to a rim at which the concave chamber terminates.

17. The mask of claim 1, the mask body comprising a unitary part manufactured from thermoplastic.

18. A method, comprising:
grasping a mask body comprising an inner surface and an outer surface, the outer surface of the mask body defining a first tang protruding from the outer surface and defining a first concave finger receiving surface and a second tang protruding from the outer surface of the mask body and defining a second concave finger receiving surface; and
placing the mask body over one or more of a mouth or a nose of a patient.

19. The method of claim 18, the grasping comprising placing a thumb in the first concave finger receiving surface and a finger in the second concave finger receiving surface.

20. The method of claim 19, further comprising grasping a chin of the patient with one or more fingers other than the thumb and the finger.
